(19) United States
(12) Patent Application Publication
(10) Pub. No.: US 2025/0213500 A1
(43) Pub. Date: Jul. 3, 2025

(54) PHARMACEUTICAL COMPOSITION FOR IMPROVING LIFE QUALITY OF ELDERLY PEOPLE ON BASIS OF VITAMIN K2, PREPARATION METHOD THEREFOR AND USE THEREOF

(71) Applicants: NANJING ASCEND BIOTECHNOLOGY INSTITUTE CO., LTD., Nanjing (CN); NANJING YISHENG KANGPIN BIOTECHNOLOGY CO., LTD., Nanjing (CN)

(72) Inventors: Tianming HE, Nanjing (CN); Luqiu SHI, Nanjing (CN); Hongyu XUE, Nanjing (CN); Guizhen SU, Nanjing (CN)

(73) Assignees: NANJING ASCEND BIOTECHNOLOGY INSTITUTE CO., LTD., Nanjing, JS (CN); NANJING YISHENG KANGPIN BIOTECHNOLOGY CO., LTD., Nanjing, JS (CN)

(21) Appl. No.: 18/850,593

(22) PCT Filed: Jun. 9, 2022
(86) PCT No.: PCT/CN2022/097816

§ 371 Date: Sep. 25, 2024

(30) Foreign Application Priority Data
Mar. 25, 2022 (CN) 202210301542.7

(51) Int. Cl. A61K 31/122; A61K 9/48; A61K 31/202; A61K 31/26; A61K 31/7012; A61K 31/7032; A61P 35/00

(52) U.S. Cl. CPC A61K 31/122; A61K 9/48; A61K 31/202; A61K 31/26; A61K 31/7012; A61K 31/7032; A61P 35/00

(57) ABSTRACT

A vitamin K2-based pharmaceutical composition for improving a quality of life in an elderly is provided, including a first component and a second component with a mass fraction ratio of (0.5-7):1. The preparation method and use of the pharmaceutical composition are also provided. The pharmaceutical composition, with the primary effects of anti-cancer and anti-senile dementia, also exhibits additional benefits such as strengthening bones, improving osteoporosis, anti-aging, antioxidant properties, enhancing memory and cognition, lowering blood lipids, and protecting the liver. Consequently, it effectively improves the quality of life for the elderly, providing health support and protection. The ingredients in the pharmaceutical composition of the present disclosure exhibit stable activity and high bioavailability, overcoming the technical limitations (including single functionality, poor absorption and utilization, significant gastrointestinal irritation, serious side effects, and poor palatability) of current elderly-oriented products. Long-term consumption of the present disclosure is devoid of any toxic or adverse effects.

### TECHNICAL FIELD

The present disclosure relates to the technical fields of pharmaceuticals, health products and foods, in particular to a vitamin K₂-based pharmaceutical composition for improving the quality of life in the elderly, and preparation method and use thereof.

### BACKGROUND

Recent research data indicate that cancer predominantly strikes individuals aged 45 and above, particularly among the middle-aged and elderly. This is attributed to the gradual decline in overall bodily functions and immune capabilities with the increase of age, with weakened immune surveillance and weakened elimination of mutated cells, making them more susceptible to external carcinogenic factors. Furthermore, cancer development often stems from years of chronic irritation and prolonged exposure to carcinogens, with a typical latency period of 10 to 30 years. Therefore, individuals who were exposed to certain carcinogens during their youth may have cancer at an old age after gradual abnormal cellular changes, contributing to the peak incidence of cancer among the elderly.

The World Health Organization states that one-third of cancers are entirely preventable; another one-third can be cured through early detection; and for the remaining one-third, existing medical measures can be used to prolong the lives, alleviate the pain, and improve the quality of life for the patients. Therefore, detection and intervention in precancerous lesions before they develop into cancer represents the ideal state of prevention.

Senile dementia, also known as Alzheimer's disease, is characterized by sluggish response, memory loss, and incoherent speech. Classified as a neurological disorder and a type of chronic illness, this disease predominantly affects the middle-aged and elderly, particularly those over 60 years old. Clinical medical surveys reveal that over 10 million people in China suffer from senile dementia, and this number continues to rise.

Early symptoms of senile dementia, such as memory loss and mental confusion, are often overlooked as consequences of inadequate sleep or other poor lifestyle habits, leading to missed opportunities for optimal treatment. Since senile dementia is irreversible, there is little hope for cure once it is contracted. The only option is to prevent it ahead of time.

Both cancer and senile dementia significantly impact the quality of life among the middle-aged and elderly.

Current anticancer medications are broadly categorized into Western medicines and Chinese medicines based on their therapeutic characteristics. Western medicines include chemotherapy and biological targeted therapies, while Chinese medicines include commonly prescribed herbal formulas and proprietary medicines. Effective medicines for delaying the progression of senile dementia include anticholinesterase inhibitors like donepezil hydrochloride, memantine hydrochloride, and rivastigmine. Additionally, non-specific medicines such as butylphthalide, oxiracetam, vitamin E, and citicoline sodium may also be administered, though their efficacy remains unproven and requires further investigation through large-scale clinical trials.

As is well known, Western medicines are often associated with significant side effects and high costs for those with proven efficacy, while Chinese medicines exhibit longer treatment cycles and significant inter-individual variability in response. Given the physiological decline and slowed metabolism in the elderly, prolonged use of these medications can irritate the gastrointestinal tract and compromise the absorption and utilization of active ingredients due to reduced bodily functions.

Evidence suggests that vitamin K₂ can actively inhibit cancer cell growth, making it a potential candidate for cancer prevention and clinical treatment. Moreover, combination of vitamin K₂ with existing chemotherapy drugs may yield better outcomes with reduced side effects. Research also demonstrates that vitamin K₂ plays a crucial role in energy production in defective mitochondria, suggesting its potential in treating Parkinson's disease, where mitochondrial dysfunction is present.

Currently, products aimed at enhancing the quality of life among the elderly often offer limited functionality, and there is a lack of products that address both cancer and senile dementia at the mechanistic level within China. The potential of vitamin K_{2,} primarily known for its bone-strengthening properties, remains untapped in this regard. As vitamin K₂ is a fat-soluble vitamin with low solubility in water, it is difficult to be absorbed by intestine after oral administration, and it is low in terms of bioavailability. Furthermore, its extreme sensitivity to light and heat often results in instability of the commercial products in their shelf life and significant loss of biological activity during prolonged storage.

Therefore, the development of a product that enhances the quality of life among the elderly, featuring stable activity, long shelf life, targeted functionality, safety, and minimal side effects, has become an urgent challenge for technicians in this field.

### SUMMARY

The objective of the present disclosure is to address the shortcomings in the existing technology and provide a vitamin K₂-based pharmaceutical composition for improving a quality of life in an elderly, overcoming the limitations of existing products, including single functionality, poor absorption and utilization, significant gastrointestinal irritation, serious side effects, and poor palatability.

In order to achieve the above objective, the present disclosure provides the following technical solutions.

In the first aspect, the present disclosure provides a vitamin K₂-based pharmaceutical composition for improving the quality of life in the elderly, which includes first component and second component, where first component exhibits anti-cancer and immune-regulatory effects, and second component exhibits targeted anti-senile dementia and neural repair effects;

A mass ratio of first component to second component is (0.5-7):1.

In some embodiments, first component includes primary active ingredients, and comprises or does not comprise secondary active ingredients, where the primary active ingredients include Ω-3 fatty acids, astaxanthin, and sulforaphane, and the secondary active ingredients include one or more selected from grape seed oil, wheat germ oil, Acer truncatum Bunge seed oil, camellia oil, and olive oil.

In other embodiments, second component includes primary active ingredients, and comprises or does not comprise secondary active ingredients, where the primary active ingredients include vitamin K_{2,} rare ginsenoside CK, and sialic acid, and the secondary active ingredients include one or more selected from tripterine, pterostilbene, resveratrol, curcumin, and piceatannol.

In some specific embodiments, the ratio of eicosapentaenoic acid (EPA) to docosahexaenoic acid (DHA) in the Ω-3 fatty acids is (0.1-4):1, and the vitamin K₂ is in the form of MK-7 with an all-trans structure.

In some preferred embodiments, the pharmaceutical composition includes the following ingredients in respective parts by mass: 10-85 parts of Ω-3 fatty acid, 3-20 parts of vitamin K₂, 0.1-4 parts of astaxanthin, 3-30 parts of sulforaphane, 1-15 parts of rare ginsenoside CK, and 1-30 parts of sialic acid.

Preferably, the pharmaceutical composition also includes an auxiliary which is an excipient, a binder, a glidant, and/or an antioxidant, where:

The excipient is ethanol or pure water, with an ethanol concentration of 40%-95%;

The binder is one or more selected from modified starch, pregelatinized starch, hydroxypropyl cellulose, sodium carboxymethyl cellulose, glycerol, gelatin, methylcellulose, ethylcellulose, hydroxypropyl methylcellulose, polyvinylpyrrolidone, mannitol, lactose, and microcrystalline cellulose;

The glidant is one or more selected from talc, silicon dioxide, magnesium stearate, rice flour, and titanium dioxide;

The antioxidant is one or more selected from rosemary extract, clove extract, cinnamon extract, Angelica dahurica extract, tea polyphenols, butylated hydroxyanisole, butylated hydroxytoluene, propyl gallate, tert-butylhydroquinone, and vitamin E.

In the first aspect, a dosage form of the pharmaceutical composition is one or more selected from tablets, powders, hard capsules, soft capsules, and suspensions.

In the second aspect, the use of vitamin K₂-based pharmaceutical composition for improving the quality of life in the elderly, as described above, is provided in dietary supplements and healthcare products.

In the third aspect, the present disclosure also provides a soft capsule containing the aforementioned vitamin K₂-based pharmaceutical composition for improving a quality of life in an elderly.

In the preferred embodiments of the third aspect, the pharmaceutical composition includes the main ingredients in respective parts by mass: 10-85 parts of Ω-3 fatty acid, 3-20 parts of Vitamin K₂, 0.1-4 parts of astaxanthin, 3-30 parts of sulforaphane, 1-15 parts of rare ginsenoside, 1-30 parts of sialic acid, 50-400 parts of soft capsule skin, and 20-200 parts of auxiliary.

In the fourth aspect, the present disclosure provides a method for preparing the aforementioned soft capsule containing the vitamin K₂-based pharmaceutical composition for improving the quality of life in the elderly. The preparation method includes two parts. The first part involves preparing sustained-release pellets from second component, and the second part involves encapsulating the sustained-release pellets with first component to form soft capsules. The preparation method includes the following steps:
1) mixing: placing second component containing vitamin K_{2,} rare ginsenoside CK, and sialic acid in a V-shaped mixer for dry mixing for 10-30 minutes, followed by passing through a 60-mesh sieve to obtain a mixture;
2) granulation: mixing the mixture obtained in the step 1) with an auxiliary evenly, followed by dry granulation method, with a particle size being controlled at 50-300 mesh to obtain granulated particles;
3) pelletization: mixing the particles obtained in the step 2) with predetermined parts by mass of an excipient, a binder, and a glidant from an auxiliary to obtain a resulting mixture, and placing the resulting mixture together with blank pellets in a granulator tray to prepare pellets with a powder layering method;
4) capsule liquid: Weighing first component comprising Ω-3 fatty acid, astaxanthin and sulforaphane, as well as an antioxidant from an auxiliary, and dissolving the antioxidant, the astaxanthin, and the sulforaphane separately in the Ω-3 fatty acid oil at 20-60° C. to obtain a mixed oil solution, allowing the mixed oil solution to stand for 4-12 hours to obtain the capsule liquid after defoaming;
5) capsule skin: Obtaining a soft capsule skin by using a general formula and preparation method; and
6) soft capsule: Injecting the capsule liquid obtained in the step 4) between two layers of the capsule skin containing the pellets obtained in the step 3), followed by pressing to obtain soft capsules containing the pellets therein.

Compared with the existing technology, the present disclosure has the following beneficial effects:

The composition in the present disclosure includes functional first component, which provides anticancer and immune-regulatory effects at the macro-organism level, and functional second component, which offers anti-senile dementia and neural repair effects at the micro-cellular level. These two levels work synergistically to achieve the prevention and treatment of cancer and senile dementia, thereby improving the quality of life for the elderly. Additionally, the composition has other beneficial effects such as strengthening bones, improving osteoporosis, anti-aging, antioxidation, enhancing memory and cognition, lowering blood lipids, and protecting the liver.

A preparation method provided in the present disclosure ensures a long-term stability of all active ingredients in the composition, particularly safeguarding the first-pass effect of active ingredients in functional second component in the oral cavity and stomach, preventing premature degradation, enhancing bioavailability, and ensuring long-lasting and stable efficacy. Long-term consumption is devoid of any toxic or adverse effects.

### DETAILED DESCRIPTION OF THE EMBODIMENTS

The technical solutions in the embodiments of the present disclosure are clearly and completely described in conjunction with the specific embodiments of the present disclosure. It is understood that the described embodiments are only a part of the embodiments of the present disclosure, instead of all embodiments. All other embodiments, which are derived by those skilled in the art from the embodiments given herein without making any creative effort, shall fall within the protection scope of the present disclosure.

The present disclosure provides a vitamin K₂-based pharmaceutical composition for improving the quality of life in the elderly, along with its preparation method. This composition primarily exhibits anticancer and anti-senile dementia effects, and also exhibits additional benefits such as strengthening bones, improving osteoporosis, anti-aging, antioxidant properties, enhancing memory and cognition, lowering blood lipids, and protecting the liver. Consequently, it effectively improves the quality of life for the elderly, providing health support and protection.

The present disclosure achieves the prevention and treatment of cancer and senile dementia which significantly impact the quality of life in the elderly, from both macro and micro levels. At the macro level, the present disclosure achieves anticancer and immune-regulatory effects through the selection of mild, non-irritating, and highly active universal ingredients. At the micro level, it achieves anti-senile dementia and neural repair effects through targeted regulation of cellular and mitochondrial metabolism and the induction of active substances. These two levels work synergistically to achieve the prevention and treatment of cancer and senile dementia, thereby improving the quality of life for the elderly. Additionally, the composition has other beneficial effects such as strengthening bones, improving osteoporosis, anti-aging, antioxidation, enhancing memory and cognition, lowering blood lipids, and protecting the liver.

In one aspect, the present disclosure provides a vitamin K₂ (MK-7)-based pharmaceutical composition for improving the quality of life in the elderly, including functional first component and functional second component, with a mass ratio of (0.5-7):1.

Ω-3 is a kind of long-chain polyunsaturated fatty acid. Existing research data indicates that Ω-3 has the effect of promoting heart health and preventing coronary artery disease. Ω-3 mainly protects the inner wall cells of blood vessels, restores vascular elasticity, dilates blood vessels, inhibits platelet aggregation, lowers blood lipids, decreases blood pressure and inhibits the thrombus formation, thus effectively preventing and treating cardiovascular and cerebrovascular diseases.

Moreover, Ω-3 prevents arrhythmia and sudden cardiac death. A clinical study on more than 11,000 patients with coronary heart disease has shown that taking 1-2 grams of Ω-3 unsaturated fatty acid every day can significantly reduce the death rate of patients with cardiovascular diseases, and especially reduce the incidence rate of sudden cardiac death by up to 45%.

There are mainly three kinds of Ω-3 fatty acids, including a-linolenic acid, eicosapentaenoic acid (EPA) and docosahexaenoic acid (DHA). EPA prevents stroke or myocardial infarction, lowers blood cholesterol and prevents arteriosclerosis. DHA, with the function of activating brain cells, promotes and coordinates the nerve circuit conduction, and maintains the normal operation of brain cells. DHA supplementation is used to improve concentration loss, learning disabilities, memory loss and senile dementia.

Additionally, Ω-3 has been shown to combat neurological disorders, prevent and fight against cancers, and exhibit anti-inflammatory effects. Therefore, the present disclosure preferentially incorporates Ω-3 fatty acid as part of a lifestyle intervention in the pharmaceutical composition, to reduce a risk of sudden cardiac death in the elderly.

Preferably, in the present disclosure, a ratio of EPA to DHA in the Ω-3 fatty acid is (0.1-4):1;

Further preferably, a content of EPA in Ω-3 fatty acid is ≥40%, and even more preferably, ≥45%.

Astaxanthin is a kind of ketocarotenoid widely found in biosphere. As a non-provitamin A carotenoid, it cannot be converted into vitamin A in animals but it has the antioxidant properties similar to carotenoids.

In one aspect, excessive oxygen free radicals are known to be the primary cause of aging and over 100 types of diseases, such as cancer, ischemic stroke, inflammation, eye diseases, diabetes, central nervous system disorders, cardiovascular diseases, as well as blood and respiratory diseases. Astaxanthin, with its unique molecular structure, can penetrate cell walls and directly eliminate intracellular oxygen free radicals, safeguarding the cellular health from the inside out and maintaining vitality.

In another aspect, the oxidation of low-density lipoprotein (LDL) in the blood is a significant contributor to atherosclerosis and hypertension. The high cholesterol intake in modern diet increases this risk. Astaxanthin can prevent LDL oxidation, elevate high-density lipoprotein, and restore vascular elasticity, thereby reducing the risk of hypertension, stroke, and heart attacks.

Preferably, the ratio of all-trans astaxanthin content to total astaxanthin content in the present disclosure is ≥70%.

Sulforaphane, an isothiocyanate derived from the hydrolysis of glucosinolates by myrosinase in plants, is abundant in cruciferous vegetables such as broccoli, cabbage mustard, and round red radish. It is a potent antioxidant and the most effective plant-derived anticancer agent found in vegetables.

Scientific research has revealed that sulforaphane exhibits broad-spectrum antitumor effects against cancers such as liver, lung, prostate, breast, rectal, colon, stomach, pancreatic, skin, leukemia, glioma, and nasopharyngeal cancers.

Its primary mechanisms are as follows: 1) Sulforaphane can protect DNA by regulating carcinogen-metabolizing enzymes, including inhibiting phase I enzymes and strongly inducing phase II enzymes. 2) Sulforaphane can induce autophagy and apoptosis, and block the cell cycle, thereby inhibiting cell proliferation; 3) Sulforaphane can inhibit basement membrane microtubule formation and endothelial cell proliferation, thus inhibiting angiogenesis within tumors, suppressing tumor metastasis, and preventing cancer progression. Furthermore, sulforaphane is inherently reactive and can react with some carcinogens, thereby inactivating them.

Therefore, the present disclosure preferably utilizes sulforaphane as one of the active ingredients in first component to provide macro-level anti-cancer protection.

Further preferably, the sulforaphane in the present disclosure is derived from broccoli seed extract with a content of ≥10%.

In summary, from a macro perspective, Ω-3 fatty acid, astaxanthin, and sulforaphane are preferably selected as the main active ingredients to constitute first component.

In view of similar effects or roles, first component comprises or does not comprise secondary active ingredients, including one or more selected from grape seed oil, wheat germ oil, Acer truncatum Bunge seed oil, camellia oil, and olive oil.

As a kind of fat-soluble vitamin, vitamin K₂ is mainly used to treat and prevent osteoporosis. According to the length of tail chain, K₂ can be divided into MK-4, MK-7, MK-8, MK-10 and other sub-categories. All the K₂ vitamins are similar in structure, with different side chain lengths. The longer the side chain, the better the absorption, the higher the biological activity, and the longer it exists in the blood. Therefore, long-chained MK-7 is considered as the best, due to that it is proved to have the highest bioavailability and a long half-life after oral administration, allowing it to exert its benefits over a longer period of time. In another aspect, MK-7 can be obtained by plant fermentation, which is considered as healthier.

Studies have demonstrated that vitamin K₂ exhibits anti-cancer effects against various tumor cells by inhibiting their growth, making it a potential method for prevention and clinical treatment of cancers. Current research indicates that the anti-cancer mechanisms of MK-7 primarily include: (1) During redox reactions involving the naphthoquinone nucleus in MK-7's structure, free radicals are generated, triggering the expression of nuclear transcription factor NK-kB. By inhibiting the expression of the anti-apoptotic gene bcl-2, it reduces the bcl-2/bax ratio, and makes mitochondrial function impaired, leading to cancer cell death. (2) MK-7 regulates different transcription factors through tyrosine kinases and phosphatases to produce anti-cancer effects. (3) MK-7 activates AP2 “USF” CREB, thereby enhancing PKA activity. Through the PKA and MAPK-mediated signaling pathways, it inhibits the proliferation and invasion of hepatocellular carcinoma cells. (4) Vitamin K2 can inhibit the proliferation and invasion of hepatocellular carcinoma cells and induce apoptosis by downregulating the Wnt/β-catenin signal. (5) It can inhibit angiogenesis.

Therefore, the present disclosure preferably selects vitamin K_{2,} particularly MK-7 form of K_{2,} as one of the active ingredients in second component.

Further preferably, the MK-7 is obtained through organic bean fermentation, with a trans structure and a purity of ≥99.5%. The trans structure of MK-7 exhibits higher bioavailability.

Ginsenosides, a kind of triterpenoid compounds, are the main active ingredients in ginseng. Currently, at least 60 types of ginsenosides have been isolated from ginseng. Research has shown that ginsenosides can extend lifespan, enhance physical strength, and treat immune dysfunction caused by radiotherapy and chemotherapy in cancer patients; the ginsenosides can also enhance the vitality of human surface cells and inhibit aging. Traditional ginsenosides are mostly prototype ginsenosides, which have a low biological activity and are difficult to be absorbed and utilized by the human body. After enzymatic catalysis and metabolism, prototype ginsenosides are converted into rare ginsenosides with smaller molecular weights, stronger liposolubility, and easier absorption and utilization by the human body. Rare ginsenosides include Rg3, Rh1, C-K, Rh2, Rg5, Rk1, Rh3, Rk2, Rh4, Rk3, aPPT, aPPD, etc.

Rare ginsenoside CK is a non-natural ginsenoside derived from the conversion of other protopanaxadiols. It is the primary metabolic product and ultimate absorption form of protopanaxadiols in the human intestine. Due to the specificity of the tetracyclic triterpenoid parent nucleus structure of ginsenosides, CK can only be obtained through gentle bioconversion methods.

Studies have shown that CK exerts anti-tumor effects on various tumors through mechanisms such as inducing apoptosis, inhibiting cell proliferation, inhibiting angiogenesis, and inhibiting metastasis. Among them, its effect on hepatocellular carcinoma has been extensively studied, with particularly prominent anti-cancer effects. Moreover, due to its low toxicity, CK is more suitable for long-term treatment than other chemotherapeutic drugs.

Therefore, the present disclosure preferably selects rare ginsenoside CK as one of the active ingredients in second component.

Further preferably, the purity of rare ginsenoside CK in the present disclosure is ≥98%.

Acetylneuraminic acid is a natural carbohydrate, accounting for over 99% of the entire sialic acid family, so it is also known as sialic acid. Its chemical name is N-acetylneuraminic acid.

Sialic acid is widely distributed in nature and has been found in animals, plants, and microorganisms. In the human body, the brain contains the highest level of sialic acid. The sialic acid content in cortex is 15 times of that in internal organs such as the liver and lungs.

Sialic acid is typically located at the termini of non-reducing oligosaccharides such as glycoproteins and glycolipids in the form of α-glycosides, serving as an essential component of cell membrane proteins and participating in various physiological functions on the cell surface.

Sialic acid promotes brain development primarily through its linkage to gangliosides SA and its role in neurotransmitters. The process of neural transmission in humans is essentially a weak electrochemical reaction. When the electric current passes, sialic acid, due to its strong negative charge, receives the electrical signal and causes Ca²⁺ to detach from gangliosides and enter presynaptic nerve terminals through ion channels, thereby opening neurotransmitter vesicles and releasing neurotransmitters. Neurotransmitters can cross the cell membrane, reach postsynaptic nerve terminals, and finally allow Na²⁺ to be transmitted. The series of processes in neural transmission are essentially charge transfers, with sialic acid playing a crucial role as the “starting gun” for this process.

Preferably, the present disclosure selects sialic acid as one of the active ingredients in second component to promote brain development, improve cognitive function in the elderly, enhance memory, and prevent and improve senile dementia.

Further preferably, the purity of sialic acid is ≥98%.

In summary, vitamin K_{2,} rare ginsenoside CK, and sialic acid are preferably selected as the main active ingredients of second component to fight against senile dementia, and repair cellular damage from a micro perspective.

Given similar effects or roles, preferably, second component comprises or does not comprise secondary active ingredients, including one or more selected from celastrol, pterostilbene, resveratrol, curcumin, or piceatannol.

The present disclosure has recognized that there are many raw materials or functional ingredients that can fight against cancer and senile dementia in this field, but these components do not necessarily produce synergistic effects through simple compounding. Some may even exhibit antagonistic effects, and their efficacy is often single-faceted. Therefore, from the comprehensive and synergistic prevention and treatment perspectives of cancer and senile dementia, the present disclosure has screened and selected the above-mentioned healthy, safe, and gentle ingredients through extensive experiments for optimal compounding, to achieve targeted multi-effect synergy at both macro and micro levels, ranging from macro-level anti-cancer and immune regulation to micro-level targeted regulation and induction of cellular and mitochondrial metabolism. These two levels work synergistically to achieve the prevention and treatment of cancer and senile dementia, thereby improving the quality of life for the elderly.

The pharmaceutical composition, as one of the specific embodiments of the present disclosure, includes the following ingredients in respective parts by mass: 10-85 parts of Ω-3 fatty acid, 3-20 parts of vitamin K₂ (MK-7), 0.1-4 parts of astaxanthin, 3-30 parts of sulforaphane, 1-15 parts of rare ginsenoside CK, and 1-30 parts of sialic acid.

In the second aspect of the present disclosure, it provides the preparation method for vitamin K₂-based pharmaceutical composition for improving the quality of life in the elderly. The preparation method includes two parts. The first part involves preparing sustained-release pellets from second component, and the second part involves encapsulating the sustained-release pellets with first component to form the soft capsules.

In a representative embodiment, the preparation method is as follows:
1) mixing: functional second component containing vitamin K_{2,} rare ginsenoside, and sialic acid are placed in a V-shaped mixer for dry mixing for 10-30 minutes, followed by passing through a 60-mesh sieve to obtain a mixture;
2) granulation: the mixture obtained in the step 1) is mixed with an auxiliary evenly, followed by dry granulation method, with a particle size being controlled at 50-300 mesh to obtain granulated particles;
3) pelletization: the particles obtained in the step 2) are mixed with predetermined parts by mass of an excipient, a binder, and a glidant from an auxiliary to obtain a resulting mixture, and the mixture is placed together with blank pellets in a granulator tray to prepare pellets with a powder layering method;
4) Capsule liquid: functional first component comprising Ω-3 fatty acid, astaxanthin and sulforaphane, as well as an antioxidant from an auxiliary are weighed, and the antioxidant, the astaxanthin, and the sulforaphane are dissolved separately in the Ω-3 oil at 20-60° C. to obtain a mixed oil solution, allowing the mixed oil solution to stand for 4-12 hours to obtain the capsule liquid after defoaming;
5) capsule skin: a soft capsule skin is obtained by using a general formula and preparation method;
6) soft capsule: a specialized equipment is used to inject the capsule liquid obtained in the step 4) between two layers of the capsule skin containing the pellets obtained in the step 3), followed by pressing with a special mold to obtain soft capsules containing the pellets therein.

For the preparation of sustained-release pallets, a main machine speed is 20-200 r/min, spray pressure is 0.1-1 MPa, feed speed is 20-100 r/min, and a spray pump speed is 10-90 r/min.

The choice of dry granulation and soft capsule skin preparation/filling processes is based on the requirements and follows methods widely recognized by those skilled in the art.

Formulation of second component into sustained-release pallets can not only enhance its stability and maintain its activity but also improve its bioavailability in the body. Encapsulating first component around second component is done due to the differing physical properties of the raw materials and for the purpose to effectively protect the active ingredients in second component from light, oxygen, and acid, thereby extending its shelf life and stability, avoiding the first-pass effect in the oral cavity, intestines and stomach, and enhancing the active effects.

In the third aspect, the use of vitamin K₂(MK-7)-based pharmaceutical composition for improving the quality of life in the elderly, as described above, is provided in dietary supplements and healthcare products.

In the fourth aspect of the present disclosure, a soft capsule containing the aforementioned pharmaceutical composition is provided. Preferably, the Vitamin K₂ (MK-7)-based pharmaceutical composition for improving the quality of life in the elderly includes the ingredients in respective parts by mass: 10-85 parts of Ω-3 fatty acid, 3-20 parts of Vitamin K₂ (MK-7), 0.1-4 parts of astaxanthin, 3-30 parts of sulforaphane, 1-15 parts of rare ginsenoside CK, 1-30 parts of sialic acid, 50-400 parts of soft capsule skin, and 20-200 parts of auxiliary.

Further preferably, auxiliary is the excipient, binder, glidant, and antioxidant.

In a representative embodiment, the excipient can be ethanol or purified water, with ethanol concentrations of 95%, 90%, 85%, 80%, 75%, 70%, 65%, 60%, 55%, 50%, 45%, or 40%;

In another embodiment, the binder is one or more selected from modified starch, pregelatinized starch, hydroxypropyl cellulose, sodium carboxymethyl cellulose, glycerol, gelatin, methylcellulose, ethylcellulose, hydroxypropyl methylcellulose, polyvinylpyrrolidone, mannitol, lactose, and microcrystalline cellulose;

In another embodiment, the glidant can be one or more selected from talc, silica, magnesium stearate, rice flour, and titanium dioxide.

In yet another embodiment, the antioxidant is one or more selected from rosemary extract, clove extract, cinnamon extract, Angelica dahurica extract, tea polyphenols, butylated hydroxyanisole, butylated hydroxytoluene, propyl gallate, tert-butylhydroquinone, and vitamin E.

The composition in the present disclosure includes functional first component, which provides anticancer and immune-regulatory effects at the macro level, and functional second component, which offers anti-senile dementia and neural repair effects at the micro level. These two levels work synergistically to achieve the prevention and treatment of cancer and senile dementia, thereby improving the quality of life for the elderly. Additionally, the composition has other effects such as strengthening bones, improving osteoporosis, anti-aging, antioxidation, enhancing memory and cognition, lowering blood lipids, and protecting the liver.

The preparation method provided by the present disclosure ensures long-term stability of the active ingredients in the composition while enhancing their bioavailability, resulting in more long-lasting and stable functional effects without any toxic or side effects from prolonged use.

The following embodiments are used to illustrate the present disclosure but do not limit its scope.

### Embodiment 1

This embodiment provides a soft capsule containing a Vitamin K2 (MK-7)-based pharmaceutical composition for improving the quality of life in the elderly, which includes, by weight percentage, the following ingredients:

Ω-3 fatty acid 46.98%, vitamin K₂ (MK-7) 0.009%, astaxanthin 1.6%, sulforaphane 2%, rare ginsenoside CK 8%, sialic acid 10%, soft capsule skin 29.99%, excipient 0.4%, binder 1%, glidant 0.02%, and antioxidant 0.02%.

The preparation method is as follows:
1) mixing: vitamin K₂, rare ginsenoside, and sialic acid (with predetermined parts by mass) are placed respectively in a V-shaped mixer for dry mixing for 10 minutes, followed by passing through a 60-mesh sieve to obtain a mixture;
2) granulation: the mixture obtained in the step 1) is mixed with an excipient and a glidant evenly, followed by dry granulation method, with a particle size being controlled at 100 mesh to obtain granulated particles;
3) pelletization: the particles obtained in the step 2) are mixed with predetermined parts by mass of an excipient, a binder, and a glidant from an auxiliary to obtain a resulting mixture, and the mixture is placed together with blank pellets in a granulator tray to prepare pellets with a powder layering method. In a preparation process, a main machine speed is 40 r/min, spray pressure is 0.2 MPa, feed speed is 30 r/min, and a spray pump speed is 20 r/min;
4) capsule liquid: functional component I containing Ω-3 fatty acid, astaxanthin and sulforaphane, as well as an antioxidant from an auxiliary are weighed, and the antioxidant, the astaxanthin, and the sulforaphane are dissolved separately in the Ω-3 oil at 30° C. to obtain a mixed oil solution, allowing the mixed oil solution to stand for 12 hours to obtain the capsule liquid after defoaming;
5) capsule skin: a soft capsule skin is obtained by using a general formula and preparation method;
6) soft capsule: a specialized equipment is used to inject the capsule liquid obtained in the step 4) between two layers of the capsule skin containing the pellets obtained in the step 3), followed by pressing with a special mold to obtain soft capsules containing the pellets therein.

### Embodiment 2

This embodiment provides a soft capsule containing a Vitamin K2 (MK-7)-based pharmaceutical composition for improving the quality of life in the elderly, which includes, by weight percentage, the following ingredients:

Ω-3 fatty acid 42.45%, vitamin K₂ (MK-7) 0.015%, astaxanthin 1.66%, sulforaphane 2%, rare ginsenoside CK 13.32%, sialic acid 11.65%, soft capsule skin 26.64%, excipient 0.67%, binder 1.5%, glidant 0.03%, and antioxidant 0.07%.

The preparation method is as follows:
1) mixing: vitamin K₂, rare ginsenoside, and sialic acid (with predetermined parts by mass) are placed respectively in a V-shaped mixer for dry mixing for 15 minutes, followed by passing through a 60-mesh sieve to obtain a mixture;
2) granulation: the mixture obtained in the step 1) is mixed with an excipient and a glidant evenly, followed by dry granulation method, with a particle size being controlled at 150 mesh to obtain granulated particles;
3) pelletization: the particles obtained in the step 2) with predetermined parts by mass of an excipient, a binder, and a glidant from an auxiliary to obtain a resulting mixture, and the mixture is placed together with blank pellets in a granulator tray to prepare pellets with a powder layering method. In a preparation process, a main machine speed is 40 r/min, spray pressure is 0.3 MPa, feed speed is 35 r/min, and a spray pump speed is 25 r/min;
4) capsule liquid: functional first component comprising Ω-3 fatty acid, astaxanthin and sulforaphane, as well as an antioxidant from an auxiliary are weighed, and the antioxidant, the astaxanthin, and the sulforaphane are dissolved separately in the Ω-3 oil at 35° C. to obtain a mixed oil solution, allowing the mixed oil solution to stand for 12 hours to obtain the capsule liquid after defoaming;
5) capsule skin: A soft capsule skin is obtained by using a general formula and preparation method; and
6) soft capsule: a specialized equipment is used to inject the capsule liquid obtained in the step 4) between two layers of the capsule skin containing the pellets obtained in the step 3), followed by pressing with a special mold to obtain soft capsules containing the pellets therein.

### Embodiment 3

This embodiment provides a soft capsule containing a Vitamin K2 (MK-7)-based pharmaceutical composition for improving the quality of life in the elderly, which includes, by weight percentage, the following ingredients:

Ω-3 fatty acid 34.24%, vitamin K₂ (MK-7) 0.014%, astaxanthin 1.43%, sulforaphane 2%, rare ginsenoside CK 28.53%, sialic acid 5.71%, soft capsule skin 25.68%, excipient 0.86%, binder 1.43%, glidant 0.06%, and antioxidant 0.06%.

The preparation method is as follows:
1) mixing: vitamin K₂, rare ginsenoside, and sialic acid (with predetermined parts by mass) are placed respectively in a V-shaped mixer for dry mixing for 20 minutes, followed by passing through a 60-mesh sieve to obtain a mixture;
2) granulation: the mixture obtained in the step 1) is mixed with an excipient and a glidant evenly, followed by dry granulation method, with a particle size being controlled at 200 mesh to obtain granulated particles;
3) pelletization: the particles obtained in the step 2) with predetermined parts by mass of an excipient, a binder, and a glidant from an auxiliary to obtain a resulting mixture, and the mixture is placed together with blank pellets in a granulator tray to prepare pellets with a powder layering method. In a preparation process, a main machine speed is 40 r/min, spray pressure is 0.2 MPa, feed speed is 30 r/min, and a spray pump speed is 20 r/min;

4) capsule liquid: functional first component comprising Ω-3 fatty acid, astaxanthin and sulforaphane, as well as an antioxidant from an auxiliary are weighed, and the antioxidant, the astaxanthin, and the sulforaphane are dissolved separately in the Ω-3 oil at 40° C. to obtain a mixed oil solution, allowing the mixed oil solution to stand for 11 hours to obtain the capsule liquid after defoaming;
5) capsule skin: a soft capsule skin is obtained by using a general formula and preparation method; and
6) soft capsule: a specialized equipment is used to inject the capsule liquid obtained in the step 4) between two layers of the capsule skin containing the pellets obtained in the step 3), followed by pressing with a special mold to obtain soft capsules containing the pellets therein.

### Embodiment 4

This embodiment provides a soft capsule containing a Vitamin K2 (MK-7)-based pharmaceutical composition for improving the quality of life in the elderly, which includes, by weight percentage, the following ingredients:

Ω-3 fatty acid 34.69%, vitamin K₂ (MK-7) 0.015%, astaxanthin 1.49%, sulforaphane 2.48%, rare ginsenoside CK 23.54%, sialic acid 9.91%, soft capsule skin 24.78%, excipient 0.99%, binder 1.98%, glidant 0.07%, and antioxidant 0.06%.

The preparation method is as follows:
1) mixing: vitamin K₂, rare ginsenoside, and sialic acid (with predetermined parts by mass) respectively in a V-shaped mixer for dry mixing for 20 minutes, followed by passing through a 60-mesh sieve to obtain a mixture;
2) granulation: the mixture obtained in the step 1) is mixed with an excipient and a glidant evenly, followed by dry granulation method, and with a particle size being controlled at 200 mesh to obtain granulated particles;
3) pelletization: the particles obtained in the step 2) are mixed with predetermined parts by mass of an excipient, a binder, and a glidant from an auxiliary to obtain a resulting mixture, and the mixture is placed together with blank pellets in a granulator tray to prepare pellets with a powder layering method. In a preparation process, a main machine speed is 435 r/min, spray pressure is 0.15 MPa, feed speed is 30 r/min, and a spray pump speed is 25 r/min;
4) capsule liquid: functional first component comprising Ω-3 fatty acid, astaxanthin and sulforaphane, as well as an antioxidant from an auxiliary are weighed, and the antioxidant, the astaxanthin, and the sulforaphane are dissolved separately in the Ω-3 oil at 40° C. to obtain a mixed oil solution, allowing the mixed oil solution to stand for 12 hours to obtain the capsule liquid after defoaming;
5) capsule skin: a soft capsule skin is obtained by using a general formula and preparation method; and
6) soft capsule: a specialized equipment is used to inject the capsule liquid obtained in the step 4) between two layers of the capsule skin containing the pellets obtained in the step 3), followed by pressing with a special mold to obtain soft capsules containing the pellets therein.

### Embodiment 5

This embodiment provides a soft capsule containing a Vitamin K2 (MK-7)-based pharmaceutical composition for improving the quality of life in the elderly, which includes, by weight percentage, the following ingredients:

(Ω-3 fatty acid 43.58%, vitamin K₂ (MK-7) 0.02%, astaxanthin 0.5%, sulforaphane 1.99%, rare ginsenoside CK 14.94%, sialic acid 11.21%, soft capsule skin 24.9%, excipient 1%, binder 1.74%, glidant 0.07%, and antioxidant 0.05%.

The preparation method is as follows:
1) mixing: vitamin K₂, rare ginsenoside, and sialic acid (with predetermined parts by mass) respectively in a V-shaped mixer for dry mixing for 25 minutes, followed by passing through a 60-mesh sieve to obtain a mixture;
2) granulation: the mixture obtained in the step 1 is mixed with an excipient and a glidant evenly, followed by dry granulation method, and with a particle size being controlled at 150 mesh to obtain granulated particles;
3) pelletization: the particles obtained in the step 2) are mixed with predetermined parts by mass of an excipient, a binder, and a glidant from an auxiliary to obtain a resulting mixture, and the mixture is placed together with blank pellets in a granulator tray to prepare pellets with a powder layering method. In a preparation process, a main machine speed is 45 r/min, spray pressure is 0.25 MPa, feed speed is 35 r/min, and a spray pump speed is 30 r/min;
4) capsule liquid: functional first component comprising Ω-3 fatty acid, astaxanthin and sulforaphane, as well as an antioxidant from an auxiliary are weighed, and the antioxidant, the astaxanthin, and the sulforaphane are dissolved separately in the Ω-3 oil at 35° C. to obtain a mixed oil solution, allowing the mixed oil solution to stand for 12 hours to obtain the capsule liquid after defoaming;
5) capsule skin: a soft capsule skin is obtained by using a general formula and preparation method; and
6) soft capsule: a specialized equipment is used to inject the capsule liquid obtained in the step 4) between two layers of the capsule skin containing the pellets obtained in the step 3), followed by pressing with a special mold to obtain soft capsules containing the pellets therein.

### Embodiment 6

This embodiment provides a soft capsule containing a Vitamin K2 (MK-7)-based pharmaceutical composition for improving the quality of life in the elderly, which includes, by weight percentage, the following ingredients:

Ω-3 fatty acid 47.72%, vitamin K2 (MK-7) 0.015%, astaxanthin 1.33%, sulforaphane 1.67%, rare ginsenoside CK 21.65%, sialic acid 8.33%, soft capsule skin 21.65%, excipient 0.83%, binder 1.67%, glidant 0.08%, and antioxidant 0.07%.

The preparation method is as follows:

1) mixing: vitamin K₂, rare ginsenoside, and sialic acid (with predetermined parts by mass) are placed respectively in a V-shaped mixer for dry mixing for 15 minutes, followed by passing through a 60-mesh sieve to obtain a mixture;
2) granulation: the mixture obtained in step 1 is mixed with an excipient and glidant evenly, followed by dry granulation method, and with a particle size being controlled at 150 mesh to obtain the granulated particles;
3) pelletization: the particles obtained in step 2) are mixed with a predetermined parts by mass of an excipient, a binder, and a glidant from an auxiliary to obtain a resulting mixture, and the mixture is then placed together with blank pellets in a granulator tray to prepare pellets with a powder layering method. In a preparation process, a main machine speed is 40 r/min, spray pressure is 0.3 MPa, feed speed is 35 r/min, and a spray pump speed is 25 r/min;
4) capsule liquid: functional first component comprising Ω-3 fatty acid, astaxanthin and sulforaphane, as well as an antioxidant from an auxiliary are weighed, and the antioxidants, the astaxanthin, and the sulforaphane are dissolved separately in the Ω-3 oil at 35° C. to obtain a mixed oil solution, allowing the mixed oil solution to stand for 12 hours to obtain the capsule liquid after defoaming;
5) capsule skin: a soft capsule skin is obtained by using a general formula and preparation method; and
6) soft capsule: a specialized equipment is used to inject the capsule liquid obtained in the step 4) between two layers of the capsule skin containing the pellets obtained in the step 3), followed by pressing with a special mold to obtain soft capsules containing the pellets therein.

### Verification Test

### Verification Test: Stability Test of the Composition Prepared According to the Present Disclosure

Soft capsules containing the pharmaceutical compositions from Embodiments 1 to 6 were designated as Experiments 1 to 6, respectively, while a commercially available vitamin K₂ (MK-7) soft capsule (containing 90 μg MK-7) served as the control group. All of them were individually sealed in brown bottles. Place them in a constant temperature and humidity box at 40±2° C. and 75%±5% RH environment, and do accelerated experiments. Samples were taken at 0, 1, 2, 3, 4, 5, 6, and 9 months to measure the MK-7 content. MK-7 content was determined by high performance liquid chromatography. The accelerated stability test results are as follows.

TABLE 1
Stability Test of Pharmaceutical Compositions

| | Time (Month) | | | | | | | |
|---|---|---|---|---|---|---|---|---|
| Content (%) | 0 | 1 | 2 | 3 | 4 | 5 | 6 | 9 |
| Embodiment | 100.21 | 100.23 | 100.18 | 100.24 | 100.19 | 100.14 | 100.16 | 100.15 |
| 1 | | | | | | | | |
| Embodiment | 10.32 | 100.33 | 100.34 | 100.29 | 100.30 | 100.23 | 100.26 | 100.22 |
| 2 | | | | | | | | |
| Embodiment | 100.27 | 100.19 | 100.21 | 100.11 | 100.26 | 100.12 | 100.23 | 100.16 |
| 3 | | | | | | | | |
| Embodiment | 100.58 | 100.49 | 100.47 | 100.41 | 100.42 | 100.39 | 100.39 | 100.41 |
| 4 | | | | | | | | |
| Embodiment | 100.19 | 100.12 | 100.11 | 100.08 | 100.04 | 100.17 | 100.12 | 100.01 |
| 5 | | | | | | | | |
| Embodiment | 100.12 | 100.13 | 100.19 | 100.12 | 100.13 | 100.12 | 100.09 | 100.11 |
| 6 | | | | | | | | |
| Control | 100.13 | 100.11 | 100.09 | 100.03 | 99.76 | 98.53 | 96.12 | 93.21 |
| group | | | | | | | | |

As evident from the results in Table 1, the MK-7 content in the soft capsules containing compositions from Embodiments 1 to 6 remained largely unchanged, demonstrating stability, whereas the MK-7 content in the control group began to decline after 3 months of acceleration. It shows that in the pharmaceutical composition containing MK-7 prepared by the preparation method of the present disclosure, the active ingredient of MK-7 can remain stable for a long time. Based on normal acceleration and conversion time calculations, the vitamin K₂-based pharmaceutical composition soft capsules for improving the quality of life in the elderly, prepared according to the method in the present disclosure, can ensure a shelf life of at least 3 years at room temperature.

### Verification Test: Animal Verification Test of the Efficacy of the Pharmaceutical Composition Prepared According to the Present Disclosure

a. Verification of the Inhibitory Effect of the Pharmaceutical Composition on Tumors

A total of 140 mice (equal number of males and females), approximately 3 months old, with a weight range of 15.41-18.47 g, were selected. Mice were subcutaneously inoculated with tumor cells, and their blood samples were drawn 24 hours later to test tumor markers and confirm the success of the mouse model establishment. A total of 126 mice confirmed with cancer (equal number of males and females) were selected and randomly divided into 6 groups, with 21 mice in each group (roughly equal number of males and females). These 6 groups were designated as Experimental Group 1, Experimental Group 2, Experimental Group 3, Experimental Group 4, Experimental Group 5, and Experimental Group 6. Experimental Group 1 received the pharmaceutical composition from Embodiment 4 once daily (10 mg/kg) in addition to normal feeding; Experimental Group 2 received the pharmaceutical composition from Embodiment 5 once daily (10 mg/kg); Experimental Group 3 received the pharmaceutical composition from Embodiment 6 once daily (10 mg/kg); Experimental Group 4 received pure MK-7 once daily (10 mg/kg); Experimental Group 5 received a known anticancer drug (sildenafil) once daily (10 mg/kg); and Experimental Group 6 received an equal weight of saline solution once daily, in addition to normal feeding. Treatment was discontinued after 15 days, and the mice were sacrificed by cervical dislocation 24 hours after discontinuation. Tumors were excised for histological examination, weighing, and recording. Tumor inhibition rate was calculated as follows: Tumor Inhibition Rate=(Average Tumor Weight in Control Group−Average Tumor Weight in Experimental Group)/Average Tumor Weight in Control Group. The experimental results are shown in Table 2.

TABLE 2
Effect of Pharmaceutical Composition on Tumor Inhibition

| Sample | Tumor Inhibition Rate |
|---|---|
| Experimental Group 1 | 60.45% |
| Experimental Group 2 | 57.51% |
| Experimental Group 3 | 48.69% |
| Experimental Group 4 | 35.67% |
| Experimental Group 5 | 40.55% |
| (Control Group) | |
| Experimental Group 6 | 0 |
| Blank Group) | |

As shown in Table 2, Experimental Groups 1 to 3 exhibited significant tumor inhibition rates compared to Control Group 5. Furthermore, Experimental Groups 1 to 3 showed better effect than Control Group 4, indicating that the compounding effect of the pharmaceutical composition of the present disclosure is superior to that of MK-7 alone.

b. Flow Cytometry Analysis of the Effect of the Pharmaceutical Composition on Cell Apoptosis

MCF-7 cancer cells in the logarithmic growth phase were digested and seeded into six-well plates at a density of 5×10⁵ cells per well. After 6 hours, the experimental groups (corresponding to Embodiments 1 to 6) were administered with the respective pharmaceutical compositions, while the negative control groups received an equal volume of culture medium. Forty-eight hours after administration, the cells were digested into single cells using trypsin, collected by centrifugation at 1000 rpm for 5 minutes, and washed with pre-cooled PBS buffer at 4° C. The single-cell suspension was prepared by resuspending the cell precipitate and centrifuged again at 1000 rpm for 5 minutes. The cells were washed again with pre-cooled PBS buffer at 4° C., resuspended into the single-cell suspension, and counted. Subsequently, it was centrifuged at 1000 rpm for 5 minutes to collect the cells. An appropriate amount of 1×Binding Buffer was added based on the cell count to adjust the cell concentration to 5×10⁶ cells/ml. A 100 μl aliquot of the cell suspension (containing 5×10⁵ cells) was mixed with 7 μl of FITC Annexin V and 7 μl of PI respectively. The mixture was gently inverted for even mixing, incubated in the dark at RT (25° C.) for 15 minutes. Then 400 μl 1× Binding Buffer was added. Cell apoptosis was detected with flow cytometry within 1 hour. The experimental results are shown in Table 3.

TABLE 3
Effect of the Pharmaceutical Composition on Cell Apoptosis

| Sample | Apoptosis rate (%) |
|---|---|
| Control | 0.21 |
| Experimental Group 1 | 15.63 |
| Experimental Group 2 | 19.76 |
| Experimental Group 3 | 21.23 |
| Experimental Group 4 | 27.89 |
| Experimental Group 5 | 25.76 |
| Experimental Group 6 | 26.64 |

The experimental results indicate that the pharmaceutical composition in the present disclosure could significantly induce apoptosis in MCF-7 cancer cells, which constitutes its mechanism of anticancer activity.

c. Effect of the Pharmaceutical Composition on Cognition

Forty healthy 14-18-month-old male SD rats were selected and randomly divided into a sham-operated control group, a model control group, and experimental groups 1-6 (corresponding to Embodiments 1-6, respectively), with 10 rats in each group. An animal model of mild cognitive impairment (MCI) in old age was established using the severe stenosis occlusion method of the rat common carotid artery. Morris water maze test was conducted 30 days after modeling to exclude the rats that did not meet the MCI criteria. The experimental groups were administered with the corresponding pharmaceutical composition from Embodiments (12 mg/kg) by intragastric gavage daily, while the sham-operated and model groups received the same dose of saline via intragastric gavage. The Morris water maze test was repeated after intragastric gavage for 30 consecutive days, to assess the learning and memory abilities of the rats.

The Morris water maze test was conducted in the rats of each group after administration of the pharmaceutical composition by intragastric gavage for 30 days, to assess their learning and memory abilities. Specifically, the following observation indicators were evaluated: \textcircled{1} Place navigation test: Average latency of the rats to find the platform. \textcircled{2} Spatial probe test: Number of times crossing the original platform location and the percentage of swimming time in the quadrant of the original platform. The experimental results are shown in Tables 4 and 5.

TABLE 4
Comparison of Mean Escape Latency in the Place Navigation
Test among Groups (X̅ ± S, s)

| Group | n | Day 2 | Day 3 | Day 4 | Day 5 |
|---|---|---|---|---|---|
| Sham-operated | 10 | 54.12 ± 1.15 | 46.15 ± 2.79 | 39.34 ± 2.46 | 20.08 ± 3.12 |
| Group | | | | | |
| Model group | 10 | 73.35 ± 3.45 | 69.43 ± 4.15 | 61.75 ± 2.75 | 56.78 ± 2.41 |
| Experimental | 10 | 63.21 ± 2.36 | 53.29 ± 3.16 | 42.49 ± 1.47 | 36.55 ± 2.28 |
| Group 1 | | | | | |
| Experimental | 10 | 65.79 ± 4.01 | 59.13 ± 2.46 | 50.15 ± 1.21 | 43.31 ± 3.42 |
| Group 2 | | | | | |
| Experimental | 10 | 69.35 ± 3.45 | 60.65 ± 2.18 | 55.11 ± 4.07 | 50.15 ± 2.21 |
| Group 3 | | | | | |
| Experimental | 10 | 53.41 ± 1.92 | 50.32 ± 3.46 | 43.11 ± 1.98 | 33.05 ± 1.48 |
| Group 4 | | | | | |
| Experimental | 10 | 58.55 ± 1.59 | 52.75 ± 1.46 | 45.44 ± 3.32 | 39.82 ± 2.01 |
| Group 5 | | | | | |
| Experimental | 10 | 56.98 ± 3.05 | 51.33 ± 2.17 | 44.56 ± 2.47 | 34.48 ± 1.68 |
| Group 6 | | | | | |

TABLE 5
Comparison of Results in the Spatial Probe Test
among Groups (X̅ ± S, s)

| | | Number of Times | Percentage of Swimming |
|---|---|---|---|
| | | Crossing the Target | Time in the Original |
| Group | n | Platform (times/min) | Platform Quadrant (%) |
| Sham-operated | 10 | 5.01 ± 1.11 | 52.36 ± 6.61 |
| Group | | | |
| Model group | 10 | 1.55 ± 0.51 | 24.21 ± 5.21 |
| Experimental | 10 | 2.85 ± 0.91 | 36.12 ± 7.51 |
| Group 1 | | | |
| Experimental | 10 | 2.15 ± 0.32 | 34.75 ± 4.92 |
| Group 2 | | | |
| Experimental | 10 | 3.05 ± 1.13 | 37.49 ± 6.53 |
| Group 3 | | | |
| Experimental | 10 | 4.42 ± 0.84 | 45.61 ± 6.54 |
| Group 4 | | | |
| Experimental | 10 | 4.11 ± 1.00 | 40.25 ± 7.55 |
| Group 5 | | | |
| Experimental | 10 | 3.55 ± 0.56 | 38.55 ± 5.96 |
| Group 6 | | | |

In the Morris water maze place navigation test and spatial probe test for learning and memory assessment, the escape latency and the number of platform crossings are closely related to vision-based spatial orientation memory and spatial location memory. The results revealed that the average latency to find the platform in the place navigation test for experimental groups 1-6 was lower than that of the model group (P<0.05). Among experimental groups 1-6, there were differences in data due to varying composition ratios, with the sham-operated group having the lowest latency (P<0.05). In the spatial probe test, the number of times crossing the original platform and the percentage of time crossing the target quadrant in the total swimming time in experimental groups 1-6 were significantly higher than those of the model group (P<0.05), with the sham-operated group showing the highest values (P<0.05).

In conclusion, the pharmaceutical composition in the present disclosure can improve learning and memory abilities in old-age MCI rats, suggesting an adjuvant therapeutic effect against senile dementia.

Any aspects not detailed in the present disclosure are deemed to be common knowledge among those skilled in the art.

Finally, it should be noted that the specific embodiments described above are intended to illustrate the technical solutions of the present disclosure rather than to limit them. Although the present disclosure has been described in detail with reference to the embodiments, those skilled in the art should understand that modifications and equivalent replacements can be made to the technical solutions of the present disclosure without departing from the spirit and scope of the technical solutions, all of which should be covered within the scope of the claims of the present disclosure.

What is claimed is:

1. A vitamin K₂-based pharmaceutical composition for improving a quality of life in an elderly, comprising a first component and a second component, wherein the first component exhibits anti-cancer and immune-regulatory effects, and the second component exhibits targeted anti-senile dementia and neural repair effects; and a mass ratio of the first component to the second component is (0.5-7):1.

2. The vitamin K₂-based pharmaceutical composition for improving the quality of life in the elderly according to claim 1, wherein the first component comprises primary active ingredients, and comprises or does not comprise secondary active ingredients, wherein the primary active ingredients comprise Ω-3 fatty acids, astaxanthin, and sulforaphane, and the secondary active ingredients comprise one or more selected from grape seed oil, wheat germ oil, Acer truncatum Bunge seed oil, camellia oil, and olive oil.

3. The vitamin K₂-based pharmaceutical composition for improving the quality of life in the elderly according to claim 1, wherein the second component comprises primary active ingredients, and comprises or does not comprise secondary active ingredients, wherein the primary active ingredients comprise vitamin K₂, rare ginsenoside CK, and sialic acid, and the secondary active ingredients comprise one or more selected from tripterine, pterostilbene, resveratrol, curcumin, and piceatannol.

4. The vitamin K₂-based pharmaceutical composition for improving the quality of life in the elderly according to claim 2, wherein a ratio of eicosapentaenoic acid (EPA) to docosahexaenoic acid (DHA) in the Ω-3 fatty acids is (0.1-4):1.

5. The vitamin K₂-based pharmaceutical composition for improving the quality of life in the elderly according to claim 3, wherein the vitamin K₂ is in a form of MK-7 with an all-trans structure.

6. The vitamin K₂-based pharmaceutical composition for improving the quality of life in the elderly according to claim 1, comprising the following main ingredients in respective parts by mass: 10-85 parts of Ω-3 fatty acid, 3-20 parts of vitamin K₂, 0.1-4 parts of astaxanthin, 3-30 parts of sulforaphane, 1-15 parts of rare ginsenoside CK, and 1-30 parts of sialic acid.

7. The vitamin K₂-based pharmaceutical composition for improving the quality of life in the elderly according to claim 1, further comprising an auxiliary, and the auxiliary is selected from an excipient, a binder, a glidant, and/or an antioxidant, wherein the excipient is ethanol or pure water, and the ethanol has an ethanol concentration of 40%-95%; the binder is one or more selected from modified starch, pregelatinized starch, hydroxypropyl cellulose, sodium carboxymethyl cellulose, glycerol, gelatin, methylcellulose, ethylcellulose, hydroxypropyl methylcellulose, polyvinylpyrrolidone, mannitol, lactose, and microcrystalline cellulose;
the glidant is one or more selected from talc, silicon dioxide, magnesium stearate, rice flour, and titanium dioxide; and
the antioxidant is one or more selected from rosemary extract, clove extract, cinnamon extract, Angelica dahurica extract, tea polyphenols, butylated hydroxyanisole, butylated hydroxytoluene, propyl gallate, tert-butylhydroquinone, and vitamin E.

8. Use of the vitamin K₂-based pharmaceutical composition for improving the quality of life in the elderly according to claim 1 in a dietary supplement and a health care product.

9. A soft capsule, comprising the vitamin K₂-based pharmaceutical composition for improving the quality of life in the elderly according to claim 1.

10. The soft capsule according to claim 9, wherein the pharmaceutical composition comprises the following main ingredients in respective parts by mass: 10-85 parts of Ω-3 fatty acid, 3-20 parts of Vitamin K₂, 0.1-4 parts of astaxanthin, 3-30 parts of sulforaphane, 1-15 parts of rare ginsenoside, 1-30 parts of sialic acid, 50-400 parts of soft capsule skin, and 20-200 parts of an auxiliary.

11. A preparation method of the soft capsule according to claim 9, wherein the soft capsule comprises the vitamin K₂-based pharmaceutical composition for improving the quality of life in the elderly, and the preparation method comprises a first part and a second part, wherein the first part comprises preparing sustained-release pellets from the second component, and the second part comprises encapsulating the sustained-release pellets with the first component to form the soft capsule, and the preparation method comprises the following steps:
1) mixing: placing the second component containing vitamin K₂, rare ginsenoside CK, and sialic acid in a V-shaped mixer for dry mixing for 10-30 minutes, followed by passing through a 60-mesh sieve to obtain a mixture;
2) granulation: mixing the mixture obtained in the step 1) with an auxiliary evenly, followed by dry granulation, with a particle size being controlled at 50-300 mesh to obtain granulated particles;
3) pelletization: evenly mixing the granulated particles obtained in the step 2) with a predetermined parts by mass of an excipient, a binder, and a glidant from an auxiliary to obtain a resulting mixture, and placing the resulting mixture and blank pellets in a granulator tray to prepare pellets by a powder layering method;
4) capsule liquid: weighing the first component comprising Ω-3 fatty acid, astaxanthin and sulforaphane, and an antioxidant from an auxiliary, and dissolving the antioxidant, the astaxanthin, and the sulforaphane separately in the Ω-3 fatty acid at 20-60° C. to obtain a mixed oil solution, and allowing the mixed oil solution to stand for 4-12 hours to obtain the capsule liquid after defoaming;
5) capsule skin: obtaining the capsule skin by using a general formula and preparation method; and
6) soft capsule: injecting the capsule liquid obtained in the step 4) between two layers of the capsule skin containing the pellets obtained in the step 3), followed by pressing to obtain the soft capsule containing the pellets therein.

12. A preparation method of the soft capsule according to claim 10, wherein the soft capsule comprises the vitamin K₂-based pharmaceutical composition for improving the quality of life in the elderly, and the preparation method comprises a first part and a second part, wherein the first part comprises preparing sustained-release pellets from the second component, and the second part comprises encapsulating the sustained-release pellets with the first component to form the soft capsule, and the preparation method comprises the following steps:
1) mixing: placing the second component containing vitamin K_{2,} rare ginsenoside CK, and sialic acid in a V-shaped mixer for dry mixing for 10-30 minutes, followed by passing through a 60-mesh sieve to obtain a mixture;
2) granulation: mixing the mixture obtained in the step 1) with an auxiliary evenly, followed by dry granulation, with a particle size being controlled at 50-300 mesh to obtain granulated particles;
3) pelletization: evenly mixing the granulated particles obtained in the step 2) with a predetermined parts by mass of an excipient, a binder, and a glidant from an auxiliary to obtain a resulting mixture, and placing the resulting mixture and blank pellets in a granulator tray to prepare pellets by a powder layering method;
4) capsule liquid: weighing the first component comprising Ω-3 fatty acid, astaxanthin and sulforaphane, and an antioxidant from an auxiliary, and dissolving the antioxidant, the astaxanthin, and the sulforaphane separately in the Ω-3 fatty acid at 20-60° C. to obtain a mixed oil solution, and allowing the mixed oil solution to stand for 4-12 hours to obtain the capsule liquid after defoaming;
5) capsule skin: obtaining the capsule skin by using a general formula and preparation method; and
6) soft capsule: injecting the capsule liquid obtained in the step 4) between two layers of the capsule skin containing the pellets obtained in the step 3), followed by pressing to obtain the soft capsule containing the pellets therein.
